(19) United States
(12) Patent Application Publication
(10) Pub. No.: US 2024/0261412 A1
(43) Pub. Date: Aug. 8, 2024

(54) NUTRACEUTICAL COMPOSITION FOR DELIVERY OF HYDROPHILIC AND HYDROPHOBIC BIOACTIVE SUBSTANCES

(71) Applicant: AKAY NATURAL INGREDIENTS PRIVATE LIMITED, Cochin, Kerala (IN)

(72) Inventors: Krishnakumar ILLATHU MADHAVAMENON, Cochin (IN); Balu Paulose MALIAKEL, Cochin (IN)

(21) Appl. No.: 18/564,462

(22) PCT Filed: May 30, 2022
(86) PCT No.: PCT/IB2022/055052

§ 371 Date: Nov. 27, 2023

(30) Foreign Application Priority Data
May 28, 2021 (IN) 202141019434

(51) Int. Cl. A61K 47/36; A61K 9/107; A61K 31/047; A61K 31/05; A61K 31/07; A61K 31/122; A61K 31/352; A61K 31/355; A61K 31/375; A61K 31/409; A61K 31/4375; A61K 31/593; A61K 33/04; A61K 33/06; A61K 33/26; A61K 33/30

(52) U.S. Cl. CPC A61K 47/36; A61K 9/107; A61K 31/047; A61K 31/05; A61K 31/07; A61K 31/122; A61K 31/352; A61K 31/355; A61K 31/375; A61K 31/409; A61K 31/4375; A61K 31/593; A61K 33/04; A61K 33/06; A61K 33/26; A61K 33/30

(57) ABSTRACT

The present disclosure relates to a nutraceutical composition and a process for its preparation. The nutraceutical composition comprises a bioactive substance, galactomannan mixture for the delivery of hydrophilic and hydrophobic bioactive substances. The nutraceutical composition of the present disclosure provides stable composition and enhances the concentration and duration of a bioactive substance in the systemic circulation and its efficacy.

### FIELD OF INVENTION

The present disclosure relates to nutraceutical composition for the oral delivery of hydrophilic and hydrophobic bioactive substances with enhanced bioavailability and its method of preparation.

### BACKGROUND

The nutritional compositions contain bioactive substances and extracts derived from naturally occurring substances such as botanicals, animals, and microorganisms generally referred to as natural agents or ingredients comprising both hydrophilic and hydrophobic substances. These natural agents may include micronutrients such as vitamins and minerals, polyphenol molecules, carotenoids, terpenes and terpenoids, alkaloids, saponins, amino acids, and the like, and extracts rich in such molecules that are generally administered together to obtain the desired health benefits.

The conventional drug delivery systems, are capable of delivering either a hydrophilic drug or a lipophilic drug and are poorly absorbed, rapidly degraded, poorly metabolized, undergo fast elimination, and consist of synthetic ingredients.

Polyphenols are a large group of phytochemicals, many of which are found in plant-based foods, such as fruits, vegetables, spices, herbs, tea, and the like that are widely consumed by humans. Polyphenols especially of the flavonoid family consisting of flavanols, flavonoids, flavones, and isoflavones, are known to have several potential health benefits because of their properties such as antioxidant and anti-inflammatory activities, anti-aging effects, and positive effects on the prevention of cancer, cardiovascular diseases, and neurodegenerative diseases. The polyphenols in food are present in the form of esters, glycosides, polymers, or aglycon polyphenols, and are generally poorly bioavailable due to their physicochemical properties such as hydrophobicity, low water solubility, rapid metabolism to inactive metabolites, hydrolysis by intestinal enzymes, microflora, and rapid elimination by the body. The positive effects of polyphenols on human health majorly depend on their mode of consumption and their bioavailability in the body.

Some formulations have been reported to increase the oral bioavailability of bioactive substances, such as nanoformulations of bioactive substances that have improved bioavailability compared to conventional dosage forms. However, these nanoformulations use several synthetic additives such as synthetic emulsifiers, excipients, and various organic solvents, which may not be desirable consumables. Moreover, the synthetic polymers used in these formulations are usually very expensive and require special types of equipment to prepare the final compositions. The use of synthetic polymers in nutraceuticals and functional foods is restricted by various regulatory authorities across the world.

The present invention provides water-soluble nutraceutical compositions of bioactive substances for food, nutraceutical, and pharmaceutical applications, employing unique compositions of naturally occurring substances such as dietary fibers, especially from food components, and using a simple process.

Therefore, there is a need for new delivery systems that are made of natural ingredients, capable of delivering the active substances to the desired site.

### OBJECTS

Some of the objects of the present disclosure, which at least one embodiment herein satisfies, are as follows:

It is an object of the present disclosure to provide a nutraceutical composition for the delivery of bioactive substances.

Another object of the present disclosure is to provide a nutraceutical composition for the oral delivery of hydrophilic and lipophilic substances.

Another object of the present disclosure is to provide a nutraceutical composition in powder form containing bioactive substances encapsulated in a gel matrix.

Another object of the present disclosure is to provide a nutraceutical composition containing hydrophilic and lipophilic substances that deliver a therapeutically effective amount of bioactive substances.

Another object of the present disclosure is to provide nutraceutical compositions of hydrophilic and lipophilic bioactive substances in powder form for oral administration.

Yet another objective of the present disclosure is to provide controlled delivery of bioactive substances, either alone or in combination.

Still another object of the present disclosure is to provide a nutraceutical composition that is devoid of synthetic polymers.

Another objective of the present disclosure is to provide a simple and economical process for the preparation of nutraceutical composition for the delivery of hydrophilic and lipophilic substances.

Another object of the present disclosure is to provide a process for the preparation of nutraceutical composition by using a water-based process.

Another object of the present disclosure is to provide a nutraceutical composition containing bioactive substances with enhanced systemic bioavailability and improved pharmacokinetics as observed with longer elimination half-life (T_{1/2}), longer circulation time, enhanced maximum concentration in plasma (Cmax), enhanced time of maximum absorption (Tmax) and enhanced area under blood plasma concentration versus time curve (AUC₀₋ₜ).

Another objective of the present disclosure is to provide a nutraceutical composition suitable for use as an additive for foods, beverages, and the like.

Still another objective of the present disclosure is to prepare water-soluble nutraceutical composition for enhancing the solubility of the bioactive hydrophilic and lipophilic substances.

Yet another objective of the present disclosure is to provide a nutraceutical composition that delivers the bioactive substances in the intestine.

Yet another objective of the present disclosure is to provide a nutraceutical composition that forms stable hydrogels impregnated with the bioactive substances, dried to powder form, and capable of forming the hydrogel when contacted with water or gastrointestinal fluid.

Further scope of applicability of the present invention will become apparent from the description given hereinafter. It should be understood that the description while indicating embodiments of the invention are given by way of illustration only since various changes and modifications within the spirit and scope of the invention will become apparent to those skilled in the art from the description. It is to be understood that the description and examples are explanatory and representative of the invention and in no way limit or restrict the invention as claimed.

### SUMMARY

The present disclosure relates to a nutraceutical composition and a process for its preparation. In an aspect, the nutraceutical composition comprising at least one bioactive substance in an amount in the range of 5 wt % to 50 wt % by the total weight of the composition; hydrolysed galactomannans in an amount in the range of 2.5 wt % to 25 wt % by the total weight of the composition; unhydrolysed galactomannans in an amount in the range of 20 wt % to 55 wt % by the total weight of the composition; and optionally emulsifying agent in an amount in the range of 5 wt % to 35 wt % by the total weight of the composition.

In another aspect, the process for the preparation of nutraceutical composition comprises mixing a predetermined amount of bioactive substance in a first fluid medium to obtain a bioactive solution and separately mixing an emulsifying agent in a second fluid medium to obtain a first mixture. The hydrolysed galactomannan is mixed with water to obtain hydrolysed galactomannan solution. The first mixture is mixed with hydrolysed galactomannan solution to obtain an emulsion. The bioactive solution is slowly added to the emulsion followed by first homogenization at a first predetermined pressure to obtain a first homogeneous mixture. The first homogeneous mixture is dried at a temperature in the range of 40° C. to 60° C. to obtain second homogeneous mixture. Separately, unhydrolysed galactomannan is mixed with water to obtain a unhydrolysed galactomannan gel. The second homogeneous mixture is mixed with unhydrolysed galactomannan gel followed by second homogenization at a second predetermined pressure for a predetermined time period to obtain third homogeneous mixture. The third homogeneous mixture is dried at a temperature in the range of 40° C. to 180° C. for a time period in the range of 60 min to 600 min to obtain nutraceutical composition.

### BRIEF DESCRIPTION OF THE ACCOMPANYING DRAWINGS

The present disclosure will now be described with the help of the accompanying drawing, in which:

FIG. 1A illustrates a photograph of the aqueous solutions of U-tRES (left) and RF-20 (right) indicating the enhanced solubility and colloidal nature;

FIG. 1B illustrates the TEM image of RF-20;

FIG. 1C illustrates DLS analysis of hydrodynamic size distribution of (i) pre-casted micelles before incorporation into the network (ii) RF-20;

FIG. 1D illustrates SEM images of (i) U-tRES (ii) FG (iii) RF-20;

FIG. 1E illustrates powder XRD diffractogram U-tRES, FG, and RF-20; Figure if illustrates FTIR spectra of FG, U-tRES, and RF-20;

FIG. 2 illustrates in vitro release of resveratrol from RF-20 and U-tRES at pH 6.5 and 2;

FIG. 3a illustrates plasma concentration versus time course for the free resveratrol upon ingestion of Capsules;

FIG. 3b illustrates plasma concentration versus time course for the free resveratrol upon ingestion of Sachet;

FIG. 3c illustrates total plasma resveratrol content measured upon enzymatic hydrolysis (β-glucosidase) versus time course upon ingestion of capsules;

FIG. 4a illustrates HR-TEM image of calcium ascorbate powder formulation (FC+) at 200 nm, the multilamellar vesicle structure is shown in the inset;

FIG. 4b illustrates FE-SEM image of multilamellar vesicles engulfed in the galactomannan hydrogel matrix of calcium ascorbate powder formulation;

FIG. 4c illustrates hydrodynamic size distribution of FC+ by DLS, zeta potential (−29.3 mV) of the particles of calcium ascorbate powder formulation in solution is shown in the inset;

FIG. 4d illustrates FTIR spectra of FG and FC+ of calcium ascorbate powder formulation;

FIG. 5 illustrates in vitro release of calcium ascorbate from FC+ tablet and granular powder form used for capsules and tablets;

FIG. 6 illustrates plasma ascorbate concentration versus time plot for unformulated plane release CAAS tablets (UF-CAAS), FC+(capsule), and FC+(tablet) followed by a single oral dose of 1000 mg;

FIG. 7a illustrates FTIR spectra of fisetin powder formulation FF-20;

FIG. 7b illustrates a powder XRD diffractogram of UF, FG, and FF-20 of fisetin powder formulation;

FIG. 7c illustrates differential scanning calorimetry of UF, FG, and FF-20 of fisetin powder formulation;

FIG. 7d illustrates SEM images of (i) UF (ii) FG and (iii) FF-20 of fisetin powder formulation;

FIG. 8 illustrates DLS and TEM analysis of fisetin powder formulation FF-20 in solution (a) and illustrates the hydrodynamic size distribution of fisetin micelle leached from FF-20 granular powder during in vitro dissolution after 1 h at pH 7.0 (possible micellar structure of fisetin in solution is depicted in the Inset) (b) illustrates Hydrodynamic size distribution of FF-20 solution obtained after ultrasound-aided dissolution in water at pH 7.0 (possible galactomannan-bound micellar structure of fisetin in solution is depicted in the Inset); (c) illustrates TEM image of FF-20;

FIG. 9a illustrates the time course of fisetin plasma concentration with the area under the curve, AUC (inset);

FIG. 9b illustrates the time course of geraldol plasma concentration with an area under the curve, AUC (inset);

### DETAILED DESCRIPTION

The invention is not limited to various embodiments given in this specification. The terms used in this specification generally have their ordinary meanings in the art, within the context of the invention, and in the specific context where each term is used. Unless otherwise defined, all technical and scientific terms used herein have the same meaning as commonly understood by one of skill in the art to which this invention pertains. Certain terms used herein are described below, or elsewhere in the specification to provide additional guidance to a practitioner regarding the description of the invention. In case of conflict, the present document, including definitions will control.

### Definitions

As used herein, the terms ‘around’, ‘about’, and ‘approximately’ shall generally mean within 20 percent, preferably within 10 percent, and most preferably within 5 percent of a given value or range. Numerical quantities given herein are approximate; meaning that the term ‘around’, ‘about’ or ‘approximately’ can be inferred if not expressly stated.

As used herein, the terms ‘preferred’ and ‘preferably’ refer to the embodiments of the invention that may afford certain benefits under certain conditions/circumstances. However, other embodiments may also be preferred, under the same or other conditions. Furthermore, the recitation of one or more preferred embodiments does not imply that the other embodiments are not useful, and is not intended to exclude other embodiments from the scope of the invention.

As used herein, the terms ‘comprising’, ‘including’, ‘having’, ‘containing’, involving’ and the like are to be understood to be open-ended, i.e., to mean including but not limited to.

As used herein, the term “polyphenols” refers to naturally occurring dietary phenolics or polyphenols that have phenolic structures and are widely found in plants. Polyphenols are chemically characterized as compounds with phenolic structural features, this group of natural products contains several sub-groups of phenolic compounds (Tsao, Chemistry and Biochemistry of Dietary Polyphenols; Nutrients 2010, 2, 1231-1246; doi:10.3390/nu2121231; incorporated by reference herein).

“Galactomannans” are high molecular weight hydrocolloidal polysaccharides built up of a β-(1-4)-D-mannan backbone with single D-galactose branches linked α-(1-6). They differ from each other mainly by the M/G ratio and fine structure. This molar ratio varies with plant origin typically in the range of 1.0-1.1/1.0, 1.6-1.8/1.0, 3.0/1.0, and 3.9-4.0/1.0 for fenugreek, guar, tara, and locust bean gums, respectively. The mannose/galactose ratio (M/G) varies depending on the plant source. A-D-Galactopyranose residues in the side chains have a fundamental effect on solubility. The water solubility of galactomannans increases with the increasing content of galactose (with decreasing M/G ratio).

D50 is the median diameter or the medium value of the particle size distribution for a group of particles in a sample, it is the value of the particle diameter at 50% in the cumulative distribution. It is one of the important parameters characterizing particle size.

Hydrophilic-lipophilic balance (HLB) represents the oil and water solubility of an emulsifier and is used to classify emulsifiers. The balance between the hydrophobic and hydrophilic properties of the molecules determines the performance of an emulsifying agent, for instance, the type of emulsion formed. The hydrophilic portion of an emulsifier could originate from a variety of groups, for example, the ionized forms of SO₄Na, COONa, and COOK. The lipophilic groups are typically saturated or unsaturated alkyl chains.

The HLB number is usually on a scale of 0-20. Lower HLB values are an indication of high oil affinity. A high HLB value, on the other hand, indicates high water-solubility. As the HLB value increases, the emulsifiers become more soluble in water, and their function changes from being W/O emulsifiers to being O/W emulsifiers.

As used herein, the term ‘bioactive substances’ refers to substances when administered will exert an effect upon a living organism, tissue, or cell to provide beneficial physiological, behavioural, and immunological effects.

As used herein, the term ‘unhydrolysed galactomannans’ refers to galactomannans that are extracted from any source without being subjected to any further process.

As used herein, the term ‘hydrolysed galactomannans’ refers to galactomannans which are obtained by hydrolysis of unhydrolysed galactomannans. The hydrolysed galactomannans have reduced molecular weight.

The present disclosure provides a nutraceutical composition for the oral delivery of hydrophilic and hydrophobic bioactive substances with enhanced bioavailability.

In an aspect of the present disclosure, there is provided a nutraceutical composition.

The nutraceutical composition comprising
a) at least one bioactive substance;
b) hydrolysed galactomannans;
c) unhydrolysed galactomannans; and
d) emulsifying agent.

In an embodiment of the present disclosure, the bioactive substance is at least one selected from hydrophilic substance and lipophilic substance.

In an embodiment of the present disclosure, the bioactive substance is present in an amount in the range of 5 wt % to 50 wt % by the total weight of the nutraceutical composition.

In an embodiment of the present disclosure, the hydrophilic substance is at least one selected from ascorbic acid, calcium ascorbate, vitamin B, catechins, chlorophyll, berberine hydrochloride, calcium, magnesium, iron, zinc, and selenium.

In an embodiment, the minerals are present in the form of complexes or salt form.

In an embodiment of the present disclosure, the lipophilic substance is at least one selected from vitamin A, vitamin D, vitamin E, vitamin K, resveratrol, fisetin, quercetin, lutein, flavonoids, polyphenols, and carotenoids.

In an embodiment of the present disclosure, the resveratrol is in the form trans-resveratrol and cis-resveratrol.

In an embodiment of the present disclosure, the hydrolysed galactomannans are obtained from fenugreek, guar gum, tara gum, and psyllium gum.

In an embodiment of the present disclosure, the hydrolysed galactomannans are present in an amount in the range of 5 wt % to 50 wt % by the total weight of the nutraceutical composition.

In an embodiment of the present disclosure, the hydrolysed galactomannans are characterized by reduced molecular weight compared to unhydrolysed galactomannans.

In an embodiment of the present disclosure, the unhydrolyzed galactomannans are obtained from fenugreek, xanthum gum, tamarind gum, tara gum, and psyllium gum.

In an embodiment of the present disclosure, the weight ratio of galactose to mannose in said unhydrolyzed galactomannans is in the ratio of 1:0.5 to 1:2. The unhydrolyzed galactomannans of these weight ratios have similar swelling or gelling behaviour which are suitable for the preparation of the nutraceutical composition of the present disclosure.

In an embodiment of the present disclosure, the unhydrolyzed galactomannans are present in an amount in the range of 20 wt % to 55 wt % by the total weight of the nutraceutical composition.

In an embodiment of the present disclosure, the weight ratio of the hydrolysed galactomannans to the unhydrolyzed galactomannans is present in the range of 1:1 to 1:14. The ratio of hydrolysed galactomannans to the unhydrolyzed galactomannans is adjusted based on the solubility of the bioactive substances, the ratio is adjusted accordingly to achieve the hydrophobic-hydrophilic balance of the nutraceutical formulation. The ratio is critical for the controlled release of bioactive substances from the formulation.

In an embodiment of the present disclosure, other hydrolysed and unhydrolysed dietary fiber polysaccharides such as glucomannans, arabinoxylans, glucan, and the like may also be used in a predetermined ratio suitable to obtain the desired viscosity, and gel characteristics with preferred release kinetics of the bioactive substances.

In an embodiment of the present disclosure, the emulsifying agent is at least one selected from the group consisting of lecithin, polysorbate, polyglycerol esters, quillaja extract, and glycerrizin.

In an embodiment of the present disclosure, the emulsifying agent is present in an amount in the range of 5 wt % to 35 wt % by the total weight of the composition.

The emulsifying agent increases the water solubility of the lipophilic bioactive substances.

In an embodiment of the present disclosure, the weight ratio of the emulsifier to the bioactive substances is in the range of 1:0.25 to 1:2.

In an embodiment of the present disclosure, the nutraceutical composition is in the form of powder, granules, syrup, tablets, capsules, and spherical beads.

In an embodiment of the present disclosure, the particle size of the powder when dissolved in water is in the range of 100 nm to 800 nm, preferably less than 400 nm, more preferably less than 200 nm.

In an embodiment of the present disclosure, wherein the D₅₀ particle size in solution is in the range of 100 nm to 150 nm.

In an embodiment of the present disclosure, the particle size of the powder in the dry form can be in the range of 20 to 150 mesh ASTM, preferably in the range of 20 to 80 mesh, more preferably in the range of 30 to 60 mesh range.

In an embodiment of the present disclosure, the powder or granules are directly compressible to tablets or can be filled into capsules along with suitable pharmaceutically suitable additives. The granular powder is also suitable for food formulations such as sachets, stick packs, chocolates, gummies, liquid shots, juices, bread spread, honey, and other dairy items.

In another aspect of the present disclosure, there is provided a process for the preparation of the nutraceutical composition.

The process is described in detail.

In a first step, a predetermined amount of bioactive substance is mixed with a first fluid medium to obtain a bioactive solution.

Separately, a predetermined amount of emulsifying agent is mixed with a second fluid medium to obtain a first mixture.

Separately, hydrolysed galactomannan is mixed with water to obtain a hydrolysed galactomannan solution.

The hydrolysed galactomannan solution is mixed with the first mixture to obtain an emulsion.

In an embodiment of the present disclosure, the first mixture (emulsifying agent solution) is mixed with the hydrolysed galactomannan solution and homogenised so that the emulsion stability and capacity to bind higher concentration of bioactive substance increase significantly.

The bioactive solution is slowly added to the emulsion followed by first homogenization at a first predetermined pressure to obtain a first homogeneous mixture.

In an embodiment of the present disclosure, the first fluid medium is at least one selected from the group consisting of water, glycerine, Quillaja extract, and aqueous ethanol.

In an embodiment of the present disclosure, the second fluid medium is at least one selected from aqueous ethanol, aqueous methanol, and aqueous acetone.

In an embodiment of the present disclosure, the first predetermined pressure is in the range of 800 bar to 1200 bar.

In a second step, the first homogeneous mixture is dried at a temperature in the range of 40° C. to 60° C. to obtain a second homogeneous mixture.

In a third step, the unhydrolysed galactomannan is mixed with water to obtain a unhydrolysed galactomannan gel.

In an embodiment of the present disclosure, the second homogeneous mixture is mixed with unhydrolysed galactomannan gel followed by second homogenization at a second predetermined pressure for a predetermined time period to obtain a third homogeneous mixture.

In an embodiment of the present disclosure, the second predetermined pressure is in the range of 450 bar to 600 bar or under sonication using an ultrasound generator of above 1000 KW capacity.

In an embodiment of the present disclosure, the predetermined time period is in the range of 1 hour to 5 hours.

In an embodiment of the present disclosure, the third homogeneous mixture is dried at a temperature in the range of 40° C. to 60° C., for a time period in the range of 60 min to 600 min to obtain nutraceutical composition.

In an embodiment, the drying is performed by vacuum drying, freeze-drying, and spray drying to obtain nutraceutical composition.

In an embodiment, wherein lipophilic bioactive substances are prepared in the form of solutions such as emulsions or liposomes, or micelles by using a suitable emulsifying agent.

In an embodiment of the present disclosure, the hydrolysed galactomannans are prepared by any of the following processes:
i. The unhydrolysed galactomannan fiber solution of 2 to 3% w/v in water is subjected to ultrasound aided heating at a temperature in the range of 50° C. to 70° C. wherein a 1000 KW ultrasound may be applied in pulses of 1 min duration for a time period of 30 min to 45 min to obtain hydrolysed galactomannan solution. The process is optimized by viscosity measurements.
ii. The unhydrolysed galactomannan fiber solution is subjected to acid hydrolysis at a temperature in the range of 60° C. to 95° C. or ultrasound sonication wherein a 1000 KW ultrasound has to be applied in pulses of 1 min duration for a time period of 30 min to 45 min to obtain hydrolysed galactomannan solution.
iii. The unhydrolysed galactomannan fiber solution is subjected to enzymatic hydrolysis at a temperature in the range of 45° C. to 50° C. at a pH of 4.5 to 5 for a time period of 1 hour to 3 hours to obtain hydrolysed galactomannan solution.

Further, ethanol is added to the hydrolysed galactomannan solution to obtain a precipitate. The obtained precipitate is washed with ethanol/water (concentration ratio) and dried to obtain hydrolysed galactomannan powder. In an embodiment, the drying is performed by vacuum drying, freeze-drying, spray drying, or any other suitable methods to obtain hydrolysed galactomannan powder.

The nutraceutical composition in powder form swells in the water at both acidic and basic ranges and disperses itself uniformly to form cloudy solutions.

The present invention employs hydrolysed galactomannans as a stabilizing agent for the hydrophobic substances in water by forming a stable hydrolysed galactomannan coating on bioactive substances that enhance its hydration and stability in water. The coating is stabilized by hydrophobic interactions and hydrogen bonding interactions between the bioactive substances and galactomannan chains. The hydrophilic galactomannan chains of hydrolysed galactomannans stabilize the bioactive substances in the blood from degradation by phagocytosis, elimination by reticuloendocytosis, or the like. The nutraceutical composition helps the longer circulation half-life and hence improved pharmacokinetics. The galactomannan-polyphenol interaction also helps to overcome the first-pass metabolism.

In an embodiment, the hydrolysed galactomannans aid the dispersibility of the composition.

The unhydrolyzed galactomannans prevent the bioactive substances from degradation by enzymes in the stomach, low pH, and intestinal glucuronidation.

Unhydrolyzed galactomannan interaction also enhances the mucoadhesive character of the particles for better interaction with the epithelial membranes of the intestine for controlled delivery of the bioactive substance.

In an embodiment, the compatibility of the composition is increased by using emulsifying agents to further increase the water solubility and stability of the hydrophobic or lipophilic polyphenolic compounds.

In an embodiment of the present disclosure, the use of lecithin can enhance the lecithin-galactomannan interactions to form stable forms in a solution which can further help the intestinal membrane permeability of galactomannan coated bioactive substances in solution for better absorption.

In another embodiment of the present disclosure, a predetermined ratio of hydrolysed and unhydrolysed galactomannans in the formulation can provide instant solubility of the formulation in water for its applications such as sachets and beverages.

In an embodiment of the present disclosure, the ratio of hydrolysed to unhydrolyzed galactomannans in the formulation can be adjusted for sustained delivery and extended lag time, which can help in intestine delivery for >3 h.

Lag time refers to the time taken for the powder to swell under gastrointestinal pH conditions and affect the release of the active substances from the powder form to the solution. Lag time can be adjusted or modulated to affect stomach release or intestinal release or even colon release. If the lag time is less i.e. 30 min to 1 hr, it can release in the stomach. The lag time has to be more if the bioactive substance is intended to release either in the intestine or into the colon.

In one embodiment, the current invention encompasses a mixture of hydrolysed galactomannans and unhydrolysed galactomannans as a delivery matrix where the hydrolysed form helps as an emulsifier and unhydrolysed one forms a gel.

In one embodiment, the hydrolysed form helps to stabilize the emulsions made with liposomes or micelles by forming a protective coating above liposomes/micelle forms. In one embodiment, the hydrophilic galactomannan chains can stabilize the emulsion in the blood from degradation by phagocytosis, elimination by reticuloendocytosis, and the like.

In one embodiment, the unhydrolyzed galactomannan forms help to form the gels which prevent the molecules or liposomes from degradation by stomach enzymes, low pH, and also intestinal glucuronidation. The gels have high water holding capacity and stability under acidic conditions.

### EXAMPLES

### Example 1

10 gm of trans-resveratrol (>98% purity) was dissolved in a 250 ml solution containing 3 g of Quillaja extract to obtain a trans-resveratrol solution. Separately 15 gm of lecithin was dissolved in ethanol/water (75:25 v/v) to obtain a lecithin solution; further, 10 gm of hydrolysed galactomannans was dissolved in 150 ml water to obtain a hydrolysed galactomannan gel; still further, 10 gm of unhydrolysed galactomannan was dissolved in 280 ml of water to obtain a unhydrolysed galactomannan gel.

The lecithin solution was mixed with hydrolysed galactomannan solution to obtain an emulsion. The trans-resveratrol solution was slowly added to the emulsion under stirring and subjected to 1100 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 550 bar for 4 hours to obtain a third homogeneous mixture.

The so obtained third homogeneous mixture was spray dried at an inlet temperature of 95° C. and an outlet temperature of 160° C. to obtain the trans-resveratrol powder. The particle size of the powder was 175 microns. The trans-resveratrol content in the powder was 20.2%.

### Example 2

42 gm of resveratrol (>98% purity) was dissolved in a 250 ml solution containing 3 g of Quillaja extract to obtain a resveratrol solution. Separately, 21 gm of polysorbate was dissolved in ethanol/water (92:8 v/v) to obtain a polysorbate solution; further, 2.5 gm of hydrolysed galactomannans was dissolved in 100 ml water to obtain a hydrolysed galactomannan gel; still further, 34.5 gm of unhydrolysed galactomannans was dissolved in 750 ml of water to obtain a unhydrolysed galactomannan gel.

The polysorbate solution was mixed with hydrolysed galactomannan solution to obtain an emulsion. The resveratrol solution was slowly added to the emulsion solution under stirring and subjected to 1200 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 600 bar for 5 hours to obtain a third homogeneous mixture. Also sonicated at 1000 KW as pulses of 1 to 5 min duration.

The so obtained third homogeneous mixture was vacuum dried at 55° C. at 690 mm of Hg pressure to obtain resveratrol powder. The particle size of the powder was microns. The resveratrol content in the powder was 40.6%.

### Example 3

42 gm of resveratrol (>98% purity) was dissolved in a 250 ml solution containing 3 g of Quillaja extract to obtain a resveratrol solution. Separately 21 gm of lecithin was dissolved in ethanol/water (92:8 v/v) to obtain a lecithin solution; further, 2.5 gm of hydrolysed galactomannans was dissolved in 100 ml water to obtain a hydrolysed galactomannan solution; still further, 34.5 gm of unhydrolysed galactomannans was dissolved in 750 ml of water to obtain a unhydrolysed galactomannan gel.

The lecithin solution was mixed with hydrolysed galactomannan solution to obtain an emulsion. The resveratrol solution was slowly added to the lecithin solution under stirring and subjected to 800 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 550 bar for 5 hours to obtain a third homogeneous mixture.

The so obtained third homogeneous mixture was vacuum dried at 55° C. at 690 mm of Hg pressure to obtain resveratrol powder. The particle size of the powder was microns. The resveratrol content in the powder was 40.6%.

### Example 4

10 gm of calcium ascorbate (>95% purity) was dissolved in ethanol/water (75:25 v/v) obtain a calcium ascorbate solution. Separately 22.5 gm of glycerrizin was dissolved in 110 ml of ethanol/water (90:10 v/v) to obtain a glycerrizin solution; further, 2.32 gm of hydrolysed galactomannans was dissolved in 100 ml water to obtain a hydrolysed galactomannan solution; still further, 30.2 gm of unhydrolysed galactomannans was dissolved in 750 ml of water to obtain a unhydrolysed galactomannan gel.

The glycerrizin solution was mixed with hydrolysed galactomannan solution to obtain an emulsion. The calcium ascorbate solution was slowly added to the emulsion under stirring and subjected to 1000 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 500 bar for 5 hours to obtain a third homogeneous mixture.

The so obtained third homogeneous mixture was vacuum dried at 55° C. at 690 mm of Hg pressure to obtain calcium ascorbate powder. The particle size of the powder was microns. The calcium ascorbate content in the powder was 40.3%.

### Example 5

35 gm of calcium ascorbate (>95% purity) was dissolved in 50 ml of water to obtain a calcium ascorbate solution. Separately, 25 gm of lecithin was dissolved in ethanol/water (90:10 v/v) to obtain a lecithin solution; further, 4 gm of hydrolysed galactomannans was dissolved in 100 ml water to obtain a hydrolysed galactomannan solution; still further, 36 gm of unhydrolysed galactomannans was dissolved in 750 ml of water to obtain a unhydrolysed galactomannan gel.

The lecithin solution was mixed with hydrolysed galactomannan solution to obtain an emulsion. The calcium ascorbate solution was slowly added to the lecithin solution under stirring and subjected to 800 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 600 bar for 4 hours to obtain a third homogeneous mixture.

The so obtained third homogeneous mixture was spray dried at an inlet temperature of 170-175° C. and an outlet temperature of 85-90° C. to obtain calcium ascorbate powder. The particle size of the powder was <250 microns. The calcium ascorbate content in the powder was 31.2%.

### Example 6

20 gm of fisetin (>95% purity) was dissolved in 250 ml of water to obtain a fisetin solution. Separately 15 gm of lecithin was dissolved in 75 ml of ethanol/water (90:10 v/v) to obtain a lecithin solution; further, 10 gm of hydrolysed galactomannans was dissolved in 200 ml of water to obtain a hydrolysed galactomannan gel; still further, 50 gm of unhydrolysed galactomannans was dissolved in 800 ml of water to obtain a unhydrolysed galactomannan gel.

The lecithin solution was mixed with hydrolysed galactomannan solution to obtain an emulsion. The fisetin solution was slowly added to the emulsion under stirring and subjected to 1000 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan gel slowly under stirring at a pressure of 600 bar for 4 hours to obtain a third homogeneous mixture.

The so obtained third homogeneous mixture was spray dried at an inlet temperature of 165-170° C. and an outlet temperature of 80-85° C. to obtain fisetin powder. The particle size of the powder was <250 microns. The fisetin content in the powder was 18.1%.

### Example 7

48 gm of fisetin (>95% purity) was dissolved in 250 ml water containing 3 gm of Quillaja extract to obtain a fisetin solution. Separately 17 gm of lecithin was dissolved in 90 ml of ethanol/water (95:5 v/v) to obtain a lecithin solution; further, 2.7 gm of hydrolysed galactomannans was dissolved in 750 ml of water to obtain a hydrolysed galactomannan gel; still further, 32.3 gm of unhydrolysed galactomannans was dissolved in 750 ml of water to obtain a unhydrolysed galactomannan gel.

The lecithin solution was mixed with hydrolysed galactomannan solution to obtain an emulsion. The fisetin solution was slowly added to the emulsion under stirring and subjected to 950 bar pressure to obtain a first homogeneous mixture containing micelles of less than 500 nm. The first homogeneous mixture was heated at 50° C. to remove ethanol and obtain a second homogeneous mixture.

The second homogeneous mixture was mixed with unhydrolysed galactomannan solution slowly under stirring at a pressure of 550 bar for 4 hours to obtain a third homogeneous mixture.

The so obtained third homogeneous mixture was vacuum dried at an inlet temperature of 60-65° C. and an outlet temperature of 55-60° C. to obtain fisetin powder. The particle size of the powder was <400 microns. The fisetin content in the powder was 45.2%.

### Example 8: Solubility of Resveratrol

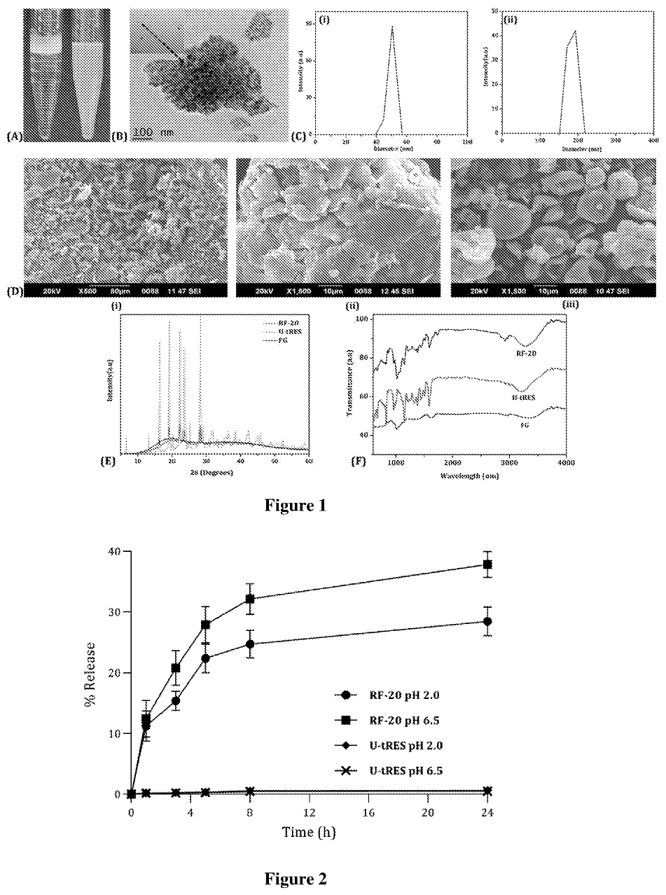

The solubility of the trans-resveratrol powder obtained in Example 1 (RF-20) and unformulated trans-resveratrol (U-tRES) was mixed with water, it was observed that RF-20 exhibited enhanced solubility, the results are depicted in FIG. 1A.

### Example 9: Surface Morphology of the Resveratrol Powder Formulation

The surface morphology of the trans-resveratrol powder obtained in Example 1 (RF-20) of the present disclosure was evaluated by using TEM. The transmission electron microscopy (TEM) analysis of the powder was performed for the structural characterization of particles in solution (JEOL JEM-2100 LaB6, Jeol Co Limited, Japan). The TEM analysis revealed that monodispersed and spherical micelles of trans-resveratrol with <40 nm were uniformly entrapped and tightly packed within the galactomannan network as aggregated particles of around 150 nm, as depicted in FIG. 1B.

The particle size analysis was evaluated by using the dynamic light scattering (DLS) method, employing Horiba SZ-100 particle size analyzer, Horiba India Private Limited, Bengaluru, India. The dynamic light scattering (DLS) particle size analysis indicated a particle size of 47.2±2.9 nm size for the pre-casted micelles before incorporation into the network, as depicted in FIG. 1C (i), and an average particle size of 172±10.4 nm for trans-resveratrol powder obtained in Example 1 (RF-20), due to the aggregation of micelles in the galactomannan hydrogel network, as depicted in FIG. 1C (ii).

The surface morphology of the trans-resveratrol powder obtained in Example 1 was analyzed by using Scanning electron microscopy (SEM) using a ZEISS Sigma 500 VP, ZEISS microscopy, Oberkochen, Germany. The SEM image of FG (hydrolysed and unhydrolyzed fenugreek galactomannan), unformulated trans-resveratrol (U-tRES), and RF-20 (hybrid-hydrogel powder of trans-resveratrol of example 1) are shown in the FIG. 1D. The crystalline nature of resveratrol, amorphous FG matrix, and spherical amorphous particles with a smooth, translucent surface morphology due to the impregnation of crystalline resveratrol into the amorphous fenugreek galactomannan matrix was clear from SEM images, depicted in FIG. 1D(i), FIG. 1D (ii) and FIG. 1D (iii) respectively.

The crystallinity trans-resveratrol powder obtained in Example 1 was further evaluated by powder X-ray diffraction (PXRD) employing a Bruker D8 Advance instrument: target Cu, k-1.54 A°, filter—Ni, voltage 40 kV, the time constant 5 min/s; scanning rate 1°/min (Bruker AXS GmbH, Karlsruhe, Germany). The powder X-ray diffraction (PXRD) indicated the 20 in the range of range 6-60°, as depicted in FIG. 1E. The unformulated trans-resveratrol (U-tRES) provided sharp and intense peaks at 2θ values (6.6, 16.4, 19.2, 22.4, 23.6, 25.3, and 28.4°), and the diffractogram for FG (fenugreek galactomannan) was characteristic of an amorphous substance. RF-20 on the other hand exhibited an amorphous nature as evident from the less intense efficiency of 92.1±1.16%, as depicted in FIG. 1E.

The entrapment of trans-resveratrol in fenugreek hydrogel and its structural integrity were confirmed by Fourier transform infrared spectrometry (FTIR) (Nicolet iS50 FTIR Spectrometer, Thermo Fisher Scientific, Massachusetts, USA) in the wavelength range 400-4000 cm⁻¹. The formation of micelle/hydrogel composites was confirmed by FTIR spectroscopy. FTIR spectra of the FG, U-tRES, and RF-20. FTIR spectrum of U-tRES showed characteristic peaks corresponding to the key structural features of trans-resveratrol as depicted in FIG. 1F. The peak observed at 3209 cm⁻¹ was corresponding to the O—H stretching of the phenolic hydroxyl groups. The stretching related to C═C bonds of the aromatic rings was visible at 1611 to 1500 cm⁻¹. The peak observed around 1147 cm⁻¹ can be attributed to the C—O stretching vibrations of phenolic groups and the phenolic O—H stretching vibrations were observed at 1381 cm⁻¹. The characteristic peaks of alkene (═C—H) were observed at 962 cm⁻¹ which confirmed the trans-configuration of the resveratrol. The stretching at 860 to 770 cm⁻¹ was characteristic of ═C—H vibration bands of arene conjugated to the olefinic group. All these peaks were found to be present in RF-20 as well, along with the characteristic peaks of FG (3200, 2914, 104, 1000-1200, 1653, 872, 800-820 cm⁻¹). This confirmed the encapsulation of resveratrol in the fenugreek galactomannan matrix without chemical modifications.

### Example 10: In Vitro Release Kinetics of Trans-Resveratrol Formulation

The in vitro release of trans-resveratrol from the powder obtained in Example 1 (RF-20) and unformulated trans-resveratrol (U-tRES) was evaluated at pH 6.5 and 2 using phosphate buffer solution and 0.1M HCl respectively, for 24 h employing a USP dissolution apparatus (Electro lab, Mumbai, India). Briefly, a 50 mg of RF-20 was dispersed in a 10 mL solution of appropriate pH and kept under constant stirring at 37±0.5° C. About 500 μL of clear solution was carefully withdrawn from the mixture at various time intervals (1, 3, 5, 8, and 24 h) and made up to 50 mL using methanol. The amount of resveratrol content in the solution was determined by HPLC, with the help of a calibration curve. The experiment was performed in triplicate and the mean cumulative percentage of released resveratrol was calculated and plotted against time to follow the release kinetics.

It is evident from the data that in vitro release of resveratrol from RF-20 indicated a sustained release under both stomach and intestinal pH conditions. The powder form of RF-20 released almost 37.9% of resveratrol at pH 6.5 and 28.5% at pH 2, in 24 h time. The unformulated resveratrol was insoluble, as depicted in FIG. 2.

### Example 11: Pharmacokinetics Study of Resveratrol

The bioavailability of the trans-resveratrol powder formulation obtained in example 1 was evaluated using a randomized, double-blinded, placebo-controlled, 2-arm, 4-sequence crossover design.

The bioavailability study in human subjects was carried out in two phases. In the first phase, identical hard shell gelatin capsules of either the trans-resveratrol powder formulation obtained in example 1 (RF-20) or unformulated resveratrol (U-tRES) containing 80 mg of trans-resveratrol were provided. Each of the formulated capsules contained 400±10 mg of trans-resveratrol powder formulation obtained in example 1 having 20.2% (w/w) of trans-resveratrol content along with another 50 mg of microcrystalline cellulose as an excipient. The unformulated capsule contained 80±5 mg of trans-resveratrol with 98.2% purity isolated from Japanese Knotweed (U-tRES) by an ethanol/water extraction process and about 300 mg of microcrystalline cellulose as an excipient. In the second phase, the same dose of either the trans-resveratrol powder formulation or the unformulated resveratrol was administered in identical stick packs, each weighing about 3 g. Maltodextrin was used as an additive to make up the sachet packs to 3 g and the participants were asked to drink one sachet with about 240 mL of water.

The double-blinded, placebo-controlled, randomized pharmacokinetic study was conducted using both capsules and sachets, to investigate the influence of delivery form on the relative bioavailability of trans-resveratrol from RF-20 and U-tRES. Each dose of either the resveratrol powder formulation or the unformulated resveratrol delivered 80±5 mg of trans-resveratrol. All the selected participants (n=16; 10 males and 6 females) completed both the phases of the study without any significant side effects or adverse events, indicating the tolerability at the tested dosage.

The blood samples were analysed by tandem mass spectrometry using QTRAP technology to detect, confirm and quantify ‘free’ resveratrol content in biomatrices.

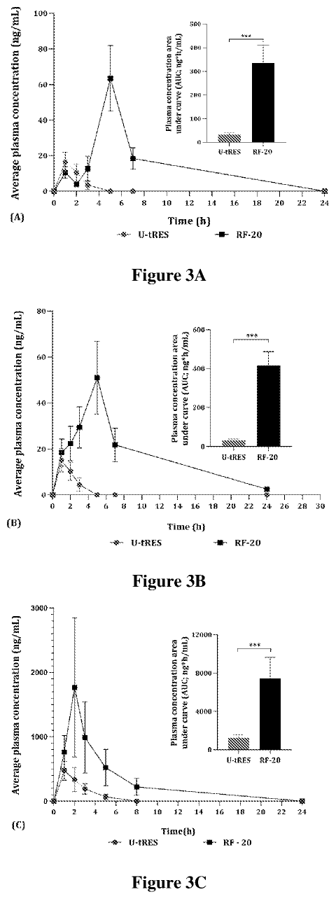

Upon the ingestion of resveratrol powder formulation (RF-20), the plasma concentration of free resveratrol and its half-life was significantly increased (***P<0.001) during 3 h to 8 h of the post-administration period, irrespective of the delivery form (FIG. 3). For capsules, the absorption maximum was at 4.86±0.53 h (Tmax), with a maximum plasma concentration (Cmax) of 63.28±16.87 ng/mL for RF-20, as compared to the Tmax of 1.07±0.26 h and Cmax of 16.34±5.67 ng/mL for the unformulated, UtRES (FIG. 3A). The area under the curve of the plasma concentration versus time plot for capsules (AUC0-24 h) for RF-20 (335.8±75.41 ng/mL·h) was 10.48-fold higher than that for the unformulated (32.05±9.97 ng/mL·h) (***P<0.001) when delivered as a capsule (Table 1). Moreover, the absorbed resveratrol from RF-20 was found to stay in the circulation for a longer duration, as evidenced by the t½ (the time taken for 50% of absorbed resveratrol to degrade) values of 6.12±1.31 h. In the case of the sachet, the Cmax was 50.97±15.82 ng/mL at a Tmax of 4.71±0.73 has compared to the Cmax of 15.07±5.10 ng/mL and Tmax of 1.21±0.42 h. The elimination half-life (t½) increased significantly (***P<0.001) from 1.58±0.65 h to 7.01±1.44 h upon formulation (FIG. 3B). It was noticed that the plasma concentration of free trans-resveratrol remained higher than about 18 ng/mL for 1 to 8 h when delivered as a sachet, indicating the sustained release property of RF-20. On the other hand, the plasma levels were undetectable after 3 h for U-tRES indicating the enhanced stability, absorption, and bioavailability of RF-20. The area under curve calculation showed a 12.98 fold enhancement for the sachet. Therefore, the bioavailability of sachet delivery was higher than the capsule form (12.98-fold versus 10.48-fold) when the AUC0-24 h was considered. The results are illustrated in Table 1 below

TABLE 1
Pharmacokinetic parameters of the unformulated trans-resveratrol
(U-tRES), and the trans-resveratrol powder formulation obtained
in example 1 RF-20, when administered as capsule and sachet
containing 80 mg trans-resveratrol per dose.

| | Pharmaco- | | | | |
|---|---|---|---|---|---|
| | kinetic | | | | |
| | Param- | Resveratrol - Capsule | | Resveratrol - Sachet | |
| | eters | U-tRES | RF-20 | U-tRES | RF-20 |
| Free | Cₘₐₓ | 16.34 ± | 63.28 ± | 15.07 ± | 50.97 ± |
| Resveratrol | (ng/mL) | 5.67 | 16.87*** | 5.10 | 15.82*** |
| | Tₘₐₓ (h) | 1.07 ± | 4.86 ± | 1.21 ± | 4.71 ± |
| | | 0.26 | 0.53* | 0.42 | 0.73* |
| | t_{1/2} (h) | 1.58 ± | 6.12 ± | 1.58 ± | 7.01 ± |
| | | 0.24 | 1.31* | 0.65 | 1.44* |
| | AUC₀₋₂₄ | 32.05 ± | 335.80 ± | 31.93 ± | 414.60 ± |
| | (ng*h/mL) | 9.97 | 75.41*** | 6.34 | 72.31*** |
| Total | Cₘₐₓ | 471.00 ± | 1768.00 ± | — | — |
| Resveratrol^{#} | (ng/mL) | 145.96 | 1080.47*** | | |
| | Tₘₐₓ (h) | 1.17 ± | 2.21 ± | — | — |
| | | 0.60 | 0.89* | | |
| | t_{1/2} (h) | 1.79 ± | 3.99 ± | — | — |
| | | 0.72 | 1.12* | | |
| | AUC₀₋₂₄ | 1254.00 ± | 7422.00 ± | — | — |
| | (ng*h/mL) | 206.30 | 1552.00*** | | |

^{#}Total resveratrol bioavailability and pharmacokinetic parameters of U-tRES and RF-20 capsule measured by treating the plasma with β-glucuronidase enzyme. U-tRES, Unformulated resveratrol; RF-20, Hybrid-FENUMAT-resveratrol formulation; Cmax, Maximum plasma concentration; tmax, Time taken to reach the maximum concentration in plasma; t½, Time taken to reduce the plasma concentration to half of its maximum observed concentration; AUC, Area under the curve. Mean values were significantly different from those of the U-tRES:
*P < 0.05,
***P < 0.001

### Example 12: Encapsulation Efficiency of Calcium Ascorbate Formulation

The encapsulation efficiency (EE) of formulation of calcium ascorbate (CAAS) was evaluated by dissolving 100 mg of CAAS obtained in Example 5 in 100 mL of water. The solution was then passed through a 0.2 μm filter and analysed by HPLC method. The column temperature was kept at 25° C. and a 20 μL sample was injected and a chromatogram was monitored at 245 nm. The encapsulation efficiency of the liposomes was calculated using the following equation:

$$Encapsulation efficiency ( EE \% ) = \frac{\text{?}}{\text{?}} \times 100 \%$$

$$\text{?} \text{indicates text missing or illegible when filed}$$

where Wt is the total amount of drug (CAAS) in the liposome suspension and Wi is the total quantity of drug added initially during preparation.

The concentration of drug loaded was determined using an HPLC method. The following equation was used to analyse the efficiency of drug loading

$$Drug load ( \% ) = \frac{( total drug - free drug )}{total drug} \times 100$$

It was observed from the HPLC results that CAAS was encapsulated into the liposomes with a high encapsulation efficiency of 95.3±0.55% and the drug loading efficiency was identified as 4.65±0.18%.

### Example 13: Characterisation of Calcium Ascorbate Formulation

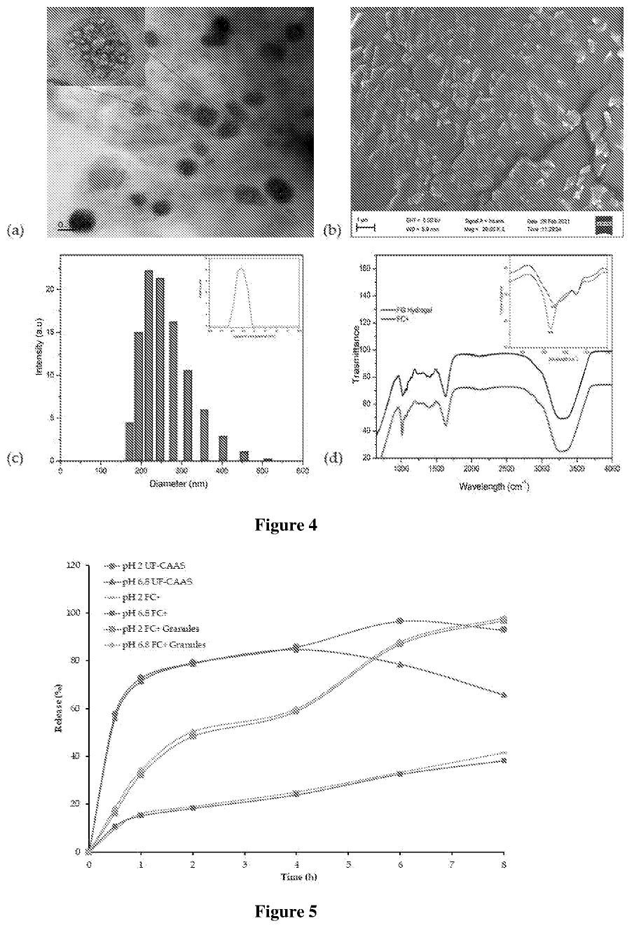

The liposomal formulation of calcium ascorbate powder formulation obtained in Example 5 (CAAS) was evaluated by electron microscopic studies (HR-TEM and FE-SEM). The calcium ascorbate formulation obtained in Example 5 is an encapsulated form of liposomal CAAS in the hydrogel matrix of fenugreek galactomannans hydrogel. The encapsulation was evident from HR-TEM image as non-spherical liposomes with a surrounding membrane structure held in the hydrophobic pockets created by the galactomannan chains in the gel phase, as depicted in FIG. 4a. The FE-SEM image also showed the uniform impregnation of non-spherical particles of about −200 nm diameter, identified as liposomes, in the gel matrix of the fibre network, as depicted in FIG. 4b. The mean particle size of encapsulated liposomal particles was observed as 212.9±5.5 nm with a zeta potential of −29.3 mV, as depicted in FIG. 4c, the mean particle size of uncoated liposomes was 89±3.9 nm with a zeta potential of −3.2 mV.

### Example 14: In Vitro Release Kinetics of Calcium Ascorbate Formulation

The in vitro release of ascorbic acid from the calcium ascorbate powder formulation was determined in water containing 1.6 mmol of the disodium salt of EDTA (ethylenediamine tetra acetic acid) at pH 2.0 and 6.8 using 0.1 M HCl and phosphate buffer respectively. 500 mg of calcium ascorbate powder formulation was mixed with 500 mL of EDTA solution to obtain the calcium ascorbate solution. The pH of the calcium ascorbate solution was adjusted and agitated at 100 rpm. About 1 mL of the solution was carefully withdrawn at regular intervals of 0, 1, 2, 4, 6, and 8 h and made up to 100 mL with water. The ascorbate content was then analysed by HPLC method. Analysis was performed in triplicate and the mean cumulative percentage of released ascorbate was calculated and plotted against time to study the in vitro release kinetics of CAAS from the fenugreek galactomannan matrix.

It is evident from the in vitro release studies that unformulated CAAS (UF-CAAS), calcium ascorbate formulation obtained in Example 5 in tablet form (FC+), and the calcium ascorbate powder formulation obtained in example 5 (FC+ Granules) indicated the sustained release of calcium ascorbate under both stomach and intestinal pH conditions, as depicted in FIG. 5. While the granular powder form released almost 85% of calcium ascorbate in 4 h, the tablets further delayed the release in such a way that only 25% release was observed in 4 h, at pH 2 and 6.8 respectively.

### Example 15: Stability Study Calcium Ascorbate Powder Formulation

The stability of the calcium ascorbate powder formulation obtained in example 5 was evaluated by accelerated stability studies as per the guidelines of International Conference on Harmonization (ICH) of technical requirements for the registration of pharmaceuticals for human use. 10 g sample packets of calcium ascorbate powder formulation were packed in polyethylene kit and kept in air-tight high-density polypropylene (HDPE) bottles and incubated at 40±2° C. and 70±5% relative humidity for a period of six months in a stability chamber (Remi, Mumbai, India). Samples were retrieved at 0, 1, 2, 3, and 6 months intervals and subjected to analysis for various parameters including colour and appearance, ascorbic acid content, moisture content, and microbial parameters (total plate counts, yeast and mould, Coliforms, Escherichia coli, and Salmonella), the results are provided below in Table 2.

TABLE 2
Accelerated stability data of FC+ powder incubated at 40 ±
2° C. and relative humidity of 70 ± 5%.

| | | | 1^{st} | 2^{nd} | 3^{rd} | 6^{th} |
|---|---|---|---|---|---|---|
| Parameter | Specification | Initial | Month | Month | Month | Month |
| Appearance | Free | Complies | Complies | Complies | Complies | Complies |
| | flowing | | | | | |
| | granular | | | | | |
| | powder | | | | | |
| Colour | Off white | Complies | Complies | Complies | Complies | Complies |
| Calcium | NLT 40% | 40.8% | 39.9% | 39.6% | 40.1% | 38.4% |
| ascorbate | | | | | | |
| content* | | | | | | |

Microbiology - USFDA (FDA)

| | | | | | | |
|---|---|---|---|---|---|---|
| Total | <10000 | 100 | 130 | 120 | 130 | 100 |
| plate | cfu/g | cfu/g | cfu/g | cfu/g | cfu/g | cfu/g |
| count^{#} | | | | | | |
| Yeast & | <200 | <10 | <10 | <10 | <10 | <10 |
| Mould^{#} | cfu/g | cfu/gm | cfu/g | cfu/g | cfu/g | cfu/g |
| Coliforms^{#} | <3 | <3 | <3 | <3 | <3 | <3 |
| | MPN/g | MPN/g | MPN/g | MPN/g | MNP/g | MPN/g |
| E. coli^{#} | Absent/g | Absent/g | Absent/g | Absent/g | Absent/g | Absent/g |
| Salmonella^{#} | Absent/25 g | Absent/25 g | Absent/25 g | Absent/25 g | Absent/25 g | Absent/25 g |

*NLT denotes ‘not less than’;
^{#}each value was presented as an average of three measurements

### Example 16: Pharmacokinetics of Calcium Ascorbate Formulation

The relative bioavailability of the calcium ascorbate formulation obtained in Example 5 (FC+) with respect to the unformulated CAAS (UF-CAAS) powder with >98% purity was investigated in healthy volunteers.

The study was designed as a randomized, double-blinded, single dose study on healthy volunteers. Healthy volunteers (males and females) (n ¼14) aged 18 to 65 years having BMI between 18-25 kgm⁻² were enrolled for the study. Those who were having a history of gut disorders such as irritable bowel syndrome, ulcerative colitis, gastro-oesophageal reflux.

Selected participants were randomized into one of the three treatment arms consisting of three single dose treatments, with either formulated (tablets and capsules) or unformulated (UFCAAS) tablets.

Tablets and hard-shell two-piece hypromellose capsules of both FC+ and UF-CAAS were prepared in such a way that each tablet weighs about 1000±25 mg and each capsule contains 500±25 mg with similar content of CAAS. The study participants were orally administered with a single dose of 1000 mg tablet or capsule as per the study protocol, along with 200±10 mL of normal drinking water.

The quantification of calcium ascorbate in plasma was carried out by a validated HPLC method.

TABLE 3
Properties of formulated and unformulated tablets
and capsules used in the present study

| | Tablets | Capsules | Tablets-Unformulated |
|---|---|---|---|
| Properties | (FC+) | (FC+) | (UF-CAAS) |
| Weight (mg) | 1000 ± 25 | 500 ± 25 | 1000 ± 25 |
| Calcium ascorbate | 403.2 ± 10 | 202.1 ± 5 | 403.5 ± 10 |
| content (mg) | | | |
| Friability (%) | 0.92 ± 0.2 | — | 1.95 ± 0.4 |
| Thickness (cm) | 0.62 ± 2 | — | 0.65 ± 2 |
| Hardness (Kg/cm²) | 5.7 ± 0.3 | — | 4.0 ± 0.5 |

A total of 25 healthy volunteers were screened; out of which 14 (10 males and 4 females) eligible participants were randomized and completed the study as per the double-blinded crossover design. The validated HPLC-PDA method of Robitaille and Hoffer was adopted for the quantification of ascorbate content in blood plasma.

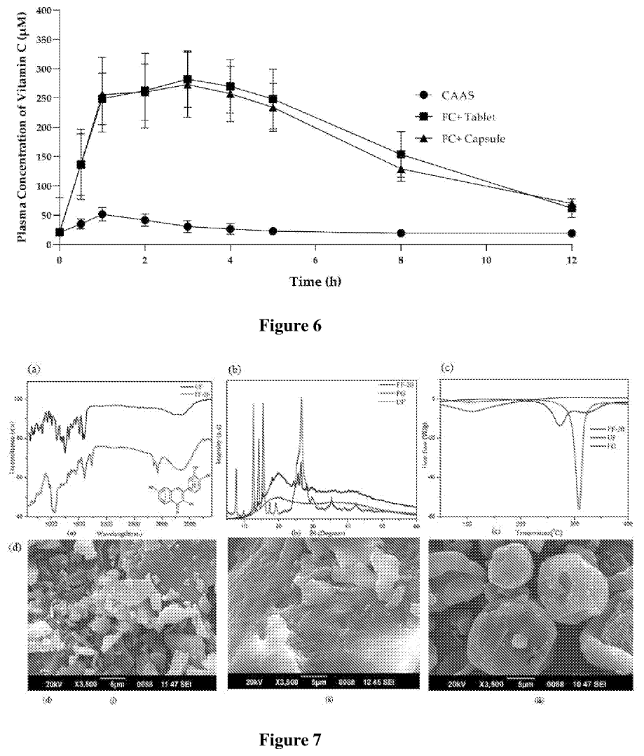

Administration of 1000 mg single dose of FC+ in both capsule and tablet form resulted in significantly (P<0.05) higher concentration of plasma ascorbate concentration as compared to the equivalent dose of UF-CAAS over 1 to 12 h of post-administration time period, as depicted in FIG. 6. Each 1000 mg of FC+ was found to contain 400±10 mg of UF-CAAS. The pharmacokinetic properties of the formulations are provided below in Table 4.

TABLE 4
Pharmacokinetic parameters of unformulated plane release CAAS tablets
(UF- CAAS), FC+ (capsule) and FC+ (tablet)

| | Dose | Cₘₐₓ | Tₘₐₓ | T_{1/2} | AUC | MRT | |
|---|---|---|---|---|---|---|---|
| Sample | (mg) | (μM) | (h) | (h) | (μM · h/mL) | (h) | Fᵣₑₗ |
| UF-CAAS | 1000 ± 25 | 51.7 ± 21 | 1 | 3.6 | 311.4 ± 63.87 | 22.67 ± 2.02 | — |
| FC+ | 1000 ± 25 | 282.4 ± 43.86 | 3 | 8.5 | 2232 ± 447.50 | 22.21 ± 1.7 | 716.76 ± 291.01 |
| (Tablets) | | | | | | | |
| FC+ | 500 ± 25 × 2 | 273 ± 59.88 | 3 | 7.6 | 2119 ± 465.19 | 22.33 ± 2.2 | 680.47 ± 288.95 |
| (Capsules) | | | | | | | |

It was evident from the above data that the area under the curve over 12 h of post-administration time period (AUC_{0-12 h}) for both tablets and capsules of FC+ were 2232 and 2119 respectively, which was about 7.2 and 6.8 times higher than the AUC₀₋₁₂ₕ of the corresponding forms of UF-CAAS, indicating 7-fold enhancement in the bioavailability of ascorbate from the formulation of CAAS (FC+). The maximum observed concentration of ascorbate in plasma (Cmax) was 282.4 and 273 mM for the FC+ tablets and capsules respectively, as compared to 51.7 mM for UF-CAAS tablet.

### Example 17: Characterization of Fisetin Powder Formulation

The FTIR analysis of unformulated fisetin (UF) showed characteristic bands at 3384 and 3254 cm⁻¹ corresponding to 0-H stretching. The aromatic C═C vibration was observed at 1510 cm⁻¹ and the bands at 1087 and 1194 cm⁻¹ were attributed to the C—O—C group vibrations. The in-plane C—H bending, C—H wagging, and O—H bending vibrations of the ring (i) & (ii) (inset in FIG. 7a) were observed in the wavelength region 1500-1300 cm−1. The C—OH stretching vibrations of the ring (ii) was observed below 1300 cm−1. In the fisetin formulation obtained in example 6 (FF-20), all the above-mentioned peaks were present along with some additional peaks characteristic of lecithin (1025 cm−1) and liposomes (2955 and 2850 cm−1), indicating the absence of any chemical modifications to fisetin. The characteristic peaks of fenugreek galactomannan (FG) were also evident at 2923 and 1055 cm−1, indicating the presence of FG, UF, and lecithin in FF-20, as depicted in FIG. 7a.

The PXRD pattern of fenugreek galactomannan (FG) provided a typical flat diffractogram corresponding to the amorphous nature of the powder. The PXRD pattern of UF indicates significant crystallinity, as evident from the multiple sharp peaks. In the fisetin formulation obtained in example 6 (FF-20), the number of peaks and their intensity were found to be significantly reduced as compared to unformulated fisetin (UF). The characteristic amorphous pattern of the galactomannan fiber was also evident in the FF-20 diffractogram, indicating the possible encapsulation of crystalline fisetin in the amorphous matrix of the galactomannan fiber network, the PXRD was depicted in FIG. 7b.

The DSC analysis showed a sharp endothermic peak at 310° C. corresponding to the melting of UF used at 220 for the formulation. Galactomannan fiber on the other hand had no characteristic peak but exhibited a 221 valley-like plot. However, FF-20 exhibited a broad less intense endothermic shift in the region of 270° C. to 320° C. with a less intense peak at 274° C., which may be attributed to the depression in the melting point of fisetin due to its extensive encapsulation effect in the fiber matrix was depicted in FIG. 7c.

The SEM image of UF, FG, and FF-20 are shown in the was depicted in FIG. 7d (i, ii, iii) respectively. The crystalline nature of fisetin was clear from FIG. 7d(i). The galactomannan fiber on the other hand was highly amorphous with no definite structure, as depicted in FIG. 7d(ii). FF-20 showed mainly the spherical form with a smooth, translucent surface, indicating the homogeneous encapsulation of fisetin into the galactomannan hydrogel matrix, as depicted in FIG. 7d(iii). It was further observed from encapsulation efficacy determination that fisetin was encapsulated with high encapsulation efficiency of 93.3±0.78% as determined by HPLC.

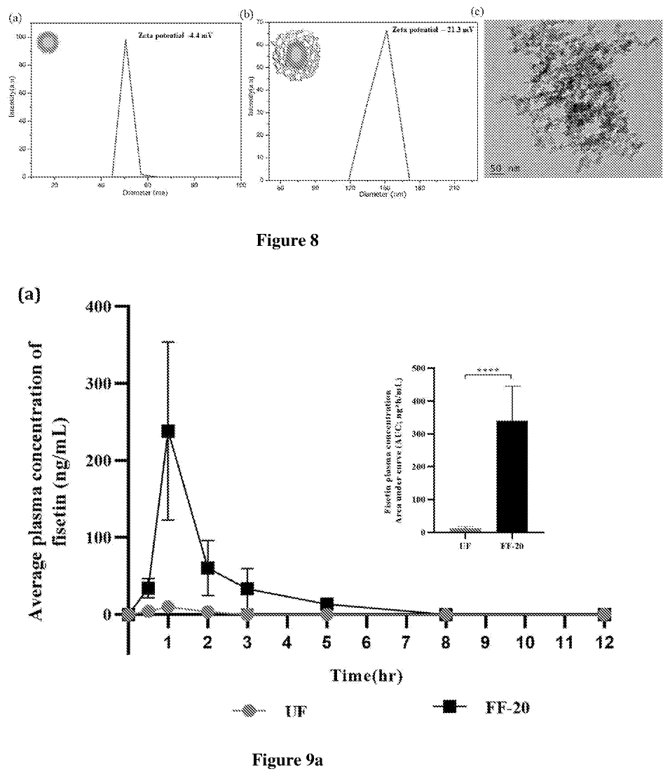

The particle size analysis of the initial micellar preparation of fisetin before impregnating into the hydrogel matrix by DLS showed uniformity and stability with an average particle size of 50±15 nm, as depicted in FIG. 8a. FF-20 solution prepared by ultra-sonication and centrifugation on the other hand showed particles of about 151.6±5.1 nm, indicating that the effective increase in the hydrodynamic volume resulted from the surface modification by the hydrophilic galactomannan chains, as depicted in FIG. 8b. Upon TEM analysis of this solution, a chain like structure was observed with relatively dark spots in various areas, as depicted in FIG. 8c. The size of these dark spots was also found to be in the range of 50 nm, possibly due to the higher concentration of the micelles in the galactomannan network as a micellar/hydrogel composite.

### Example 18: Pharmacokinetics of Fisetin Powder Formulation

The bioavailability of the fisetin formulation was evaluated in healthy human subjects. It has been evaluated in a single dose, randomized, comparative, double-blinded crossover design was adopted to evaluate the difference in the bioavailability and pharmacokinetic parameters (Cmax, AUC, Tmax, and t½) of free (unconjugated) fisetin and geraldol following the oral administration of a fisetin formulation obtained in example 6 (FF-20) and unformulated fisetin (UF). The participants were provided with a capsule contained 500±25 mg of UF or FF-20. Around 50 mg of microcrystalline cellulose was present in each capsule as a flow-improving excipient. Following administration, blood samples (2 mL) were collected 5 min (t0) prior to oral dosing and subsequently at 0.5, 1, 2, 3, 5, 8, and 12 h after oral ingestion of UF or FF-20, using an indwelling venous cannula. Participants were provided with a standardized breakfast, lunch, snack, and dinner respectively at 1, 4, 8, and 12 h after dosing. Plasma was separated by centrifugation at 11,950×g for 10 min at 4° C. and stored for a maximum of two days at −20° C. for analysis. Fisetin and geraldol in plasma samples were extracted and subjected to UPLC-ESI-MS/MS analyses.

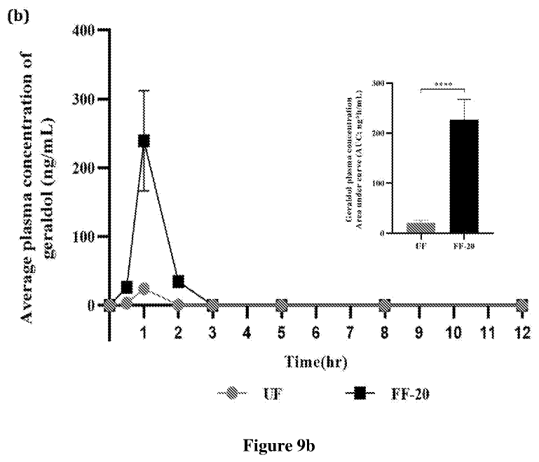

Due to the low bioavailability, detection of the unformulated fisetin in individuals was only quantifiable when supplemented at 1000 mg dose, which was equivalent to approximately five times more fisetin content than FF-20 (1000 mg of FF-20 contained only 192 mg fisetin). All results shown were adjusted for the difference in fisetin content. The time course for the plasma fisetin concentration, as depicted in FIG. 9a, was significantly higher (****P<0.0001) at all the time points detected (0.5, 1, 2, 3, 5 and 8 h post supplementation) when individuals were administered with FF-20. Fisetin levels were quantifiable up to 8 h after dosing with FF-20 compared to only 2 h after dosing with UF. The average plasma concentration of fisetin determined in the individuals when supplemented with FF-20 (AUC0-12 h=341.4 ng*h/mL) was 26.9-fold greater than when supplemented with UF (AUC0-12 h=12.67 ng*h/mL), as depicted in FIG. 9a (inset). The Cmax for fisetin, was 238.2 ng/mL at a Tmax of 1.24 h for FF-20 and that was only 9.97 ng/mL with a Tmax of 0.88 h for UFR The t½ was extended to 1.51 h when supplemented with FF-20 compared to the t½ of 1.14 h for UF (Table 5).

TABLE 5
Pharmacokinetic parameters for fisetin and geraldol: Cmax, Tmax, t½, and AUC values,
which were normalized to adjust for the higher fisetin intake in the unformulated fisetin (UF).

| | | | | | Geraldol/Fisetin | |
|---|---|---|---|---|---|---|
| | Fisetin | | Geraldol | | Ratio | |
| Groups | UF | FF-20 | UF | FF-20 | UF | FF-20 |
| Cₘₐₓ (ng/mL) | 9.97 ± 3.97 | 238.2 ± 87.26**** | 24.0 ± 7.69 | 239.23 ± 94.09*** | — | — |
| tₘₐₓ (h) | 0.88 ± 0.18 | 1.24 ± 0.35* | 0.9 ± 0.12 | 1.13 ± 0.25* | — | — |
| t_{1/2} (h) | 1.14 ± 0.09 | 1.51 ± 0.05* | 1.18 ± 0.08 | 1.59 ± 0.1* | — | — |
| AUC₀₋₁₂ | 12.67 ± 4.86 | 341.4 ± 130.05**** | 20.48 ± 6.19 | 227.14 ± 89.71**** | 1.62 | 0.67 |
| (ng*h/mL) | | | | | | |

UF, Unformulated fisetin; FF-20, Hybrid-FENUMAT-fisetin formulation; Cmax, Maximum plasma concentration; tmax, Time taken to reach the maximum concentration in plasma; t½, Time taken to reduce the plasma concentration to half of its maximum observed concentration; AUC, Area under the curve. Mean values were significantly different from those of the UF:
*P < 0.05,
***P < 0.001,
****P < 0.0001.

### Pharmacokinetics of Geraldol

In addition to evaluating the time course for fisetin in the plasma, geraldol which is an active methoxylated metabolite of fisetin was also analyzed to understand its conversion in humans. The time course for the geraldol plasma concentration, depicted in FIG. 9b, was also significantly higher (****P<0.0001) at all the time points detected (0.5, 1, 2, and 3 h post fisetin supplementation) when individuals were provided with FF-20 compared to UF. The plasma concentration of geraldol was quantifiable only up to 2 h after dosing with UF. The average plasma concentration of geraldol (FIG. 9b inset) in the individuals, when supplemented with FF-20 (AUC0-12 h=227.14 ng*h/mL), was 11.1-fold greater than when supplemented with UF (AUC0-12 h=20.48 ng*h/mL). Additionally, the AUC ratio of geraldol to fisetin after supplementation with FF-20 was 0.67, which was more than two times lower than after supplementation with UF (1.62) (Table 5 above).

The Cmax, Tmax, and t½ of geraldol also followed a similar pattern to fisetin. The Cmax for geraldol occurred at 0.9 h after dosing with UF and was shifted to 1.14 h for FF-20. The Cmax for FF-20 was 239.2 ng/mL compared to the UF plasma samples (24.0 ng/mL). The Cmax ratio of geraldol to fisetin was 2.4-fold higher in the UF plasma samples but was about the same in the FF-20 plasma samples. The t½ for UF was also slightly extended to 1.59 h in individuals when supplemented with FF-20 compared to 1.18 h for UF.

1. A nutraceutical composition comprising
a. at least one bioactive substance in an amount in the range of 5 wt % to 50 wt % by the total weight of the composition;
b. hydrolysed galactomannans in an amount in the range of 2.5 wt % to 25 wt % by the total weight of the composition;
c. unhydrolysed galactomannans in an amount in the range of 20 wt % to 55 wt % by the total weight of the composition;
d. emulsifying agent in an amount in the range of 5 wt % to 35 wt % by the total weight of the composition.

2. The composition as claimed in claim 1, wherein said bioactive substance is at least one selected from hydrophilic substance and lipophilic substance.

3. The composition as claimed in claim 2, wherein said hydrophilic substance is at least one selected from ascorbic acid, calcium ascorbate, vitamin B, catechins, chlorophyll, berberine hydrochloride, calcium, magnesium, iron, zinc, and selenium.

4. The composition as claimed in claim 2, wherein said lipophilic substance is at least one selected from the group consisting of vitamin A, vitamin D, vitamin E, vitamin K, resveratrol, fisetin, quercetin, and lutein.

5. The composition as claimed in claim 1, wherein said hydrolysed galactomannans are obtained from fenugreek, guar gum, tara gum, and psyllium gum.

6. The composition as claimed in claim 1, wherein said unhydrolyzed galactomannans are obtained from fenugreek, xanthum gum, tamarind gum, tara gum, and psyllium gum.

7. The composition as claimed in claim 5, wherein the weight ratio of galactose to mannose in said unhydrolyzed galactomannans is in the ratio of 1:0.5 to 1:2.

8. The composition as claimed in claim 1, wherein the weight ratio of said hydrolysed galactomannans to the unhydrolyzed galactomannans is in the range of 1:1 to 1:14.

9. The composition as claimed in claim 1, wherein said emulsifying agent is at least one selected from the group consisting of lecithin, polysorbate, polyglycerol esters, quillaja extract, and glycerrizin.

10. The composition as claimed in claim 1, wherein the weight ratio of emulsifying agent to the bioactive substance is in the range of 1:0.25 to 1:2.

11. The composition as claimed in claim 1, wherein said nutraceutical composition is in the form selected from powder, granules, syrup, tablets, capsules, and spherical beads.

12. The composition as claimed in claim 11, wherein the particle size of said powder in dry form is in the range of 20 to 150 mesh ASTM.

13. The composition as claimed in claim 11, wherein the particle size of said powder when dissolved in water is in the range of 100 nm to 800 nm, preferably less than 400 nm, more preferably less than 200 nm.

14. The composition as claimed in claim 11, wherein the particle size of said powder when dissolved in water, the D₅₀ particle size is in the range of 100 nm to 150 nm.

15. The composition as claimed in claim 1, wherein said composition further comprises glucomannans, arabinoxylans, and glucan.

16. A process for the preparation of nutraceutical composition as claimed in claim 1, wherein said process comprises the following steps:
a. mixing a predetermined amount of bioactive substance in a first fluid medium to obtain a bioactive solution;
b. separately mixing an emulsifying agent in a second fluid medium to obtain a first mixture;
c. separately mixing the hydrolysed galactomannan with water to obtain a hydrolysed galactomannan solution;
d. mixing said first mixture with said hydrolysed galactomannan solution to obtain an emulsion;
e. adding said bioactive solution slowly to said emulsion followed by first homogenization at a first predetermined pressure to obtain a first homogeneous mixture;
f. drying said first homogeneous mixture at a temperature in the range of 40° C. to 60° C. to obtain a second homogeneous mixture;
g. separately mixing the unhydrolysed galactomannan with water to obtain a unhydrolysed galactomannan gel;
h. mixing said second homogeneous mixture with unhydrolysed galactomannan gel followed by second homogenization at a second predetermined pressure for a predetermined time period to obtain a third homogeneous mixture; and
i. drying said third homogeneous mixture at a temperature in the range of 40° C. to 180° C. for a time period in the range of 60 min to 600 min to obtain nutraceutical composition.

17. The process as claimed in claim 16, wherein said first fluid medium is at least one selected from the group consisting of water, glycerine, Quillaja extract, and aqueous ethanol.

18. The process as claimed in claim 16, wherein said second fluid medium is at least one selected from aqueous ethanol, aqueous methanol, and aqueous acetone.

19. The process as claimed in claim 16, wherein said first predetermined pressure is in the range of 800 bar to 1200 bar.

20. The process as claimed in claim 16, wherein said second predetermined pressure is in the range of 450 bar to 600 bar; said predetermined time period is in the range of 1 hour to 5 hours.
